(19) United States
(12) Patent Application Publication
(10) Pub. No.: US 2004/0267015 A1
(43) Pub. Date: Dec. 30, 2004

(54) Peptide deformylase inhibitors

(76) Inventors: Ajita Bhat, Collegeville, PA (US); Siegfried B. Christensen, Collegeville, PA (US); James S. Frazee, King of Prussia, PA (US); Martha S. Head, Collegeville, PA (US); Jack D. Leber, King of Prussia, PA (US); Mei Li, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/473,104

(22) PCT Filed: Apr. 4, 2002
(86) PCT No.: PCT/US02/10506

Related U.S. Application Data
(60) Provisional application No. 60/281,613, filed on Apr. 5, 2001.

(51) Int. Cl. C07D 271/12; C07D 285/10; C07D 277/04; C07C 259/04; C07D 207/46

(52) U.S. Cl. 544/333; 546/336; 548/131; 548/134; 548/200; 548/215; 548/225; 548/240; 548/269.4; 548/577; 562/621

(57) ABSTRACT

PDF inhibitors and novel methods for their use are provided.

### FIELD OF THE INVENTION

The present invention relates to the use of novel anti-bacterial compounds, and pharmaceutical compositions containing these compounds as peptide deformylase inhibitors.

### BACKGROUND OF THE INVENTION

Bacterial initiator methionyl tRNA is modified by methionyl tRNA formyltransferase (FMT) to produce formyl-methionyl tRNA. The formyl methionine (f-met) is then incorporated at the N-termini of newly synthesized polypeptides. Polypeptide deformylase (PDF or Def) then deformylates primary translation products to produce N-methionyl polypeptides. Most intracellular proteins are further processed by methionine amino peptidase (MAP) to yield the mature peptide and free methionine, which is recycled. PDF and MAP are both essential for bacterial growth, and PDF is required for MAP activity. This series of reactions is referred to as the methionine cycle (FIG. 1).

[image]

To date, polypeptide deformylase homologous genes have been found in bacteria, in chloroplast-containing plants, in mice and in human. The plant proteins are nuclear encoded but appear to carry a chloroplast localisation signal. This is consistent with the observation that chloroplast RNA and protein synthesis processes are highly similar to those of eubacteria. While there is limited information on protein expression of mammalian PDF gene homologs (Bayer Aktiengesellschaft, Pat. WO2001/42431), no functional role for such proteins has been demonstrated to date (Meinnel, T., Parasitology Today 16(4), 165-168, 2000).

Polypeptide deformylase is found in all eubacteria for which high coverage genomic sequence information is available. Sequence diversity among PDF homologs is high, with as little as 20% identity between distantly related sequences. However, conservation around the active site is very high, with several completely conserved residues, including one cysteine and two histidines which are required to coordinate the active site metal (Meinnel, T. et al., J. Mol. Biol. 267, 749-761, 1997).

PDF is recognized to be an attractive antibacterial target, as this enzyme has been demonstrated to be essential for bacterial growth in vitro (Mazel, D. et al., EMBO J. 13 (4), 914-923, 1994), is not believed to be involved in eukaryotic protein synthesis (Rajagopalan et al., J. Am. Chem. Soc. 119, 12418-12419, 1997), and is universally conserved in prokaryotes (Kozak, M., Microbiol. Rev. 47, 1-45, 1983). Therefore PDF inhibitors can potentially serve as broad spectrum antibacterial agents.

### SUMMARY OF THE INVENTION

The present invention involves novel anti-bacterial compounds represented by Formula (I) hereinbelow and their use as PDF inhibitors.

The present invention further provides methods for inhibiting PDF in an animal, including humans, which comprises administering to a subject in need of treatment an effective amount of a compound of Formula (I) as indicated hereinbelow.

### DETAILED DESCRIPTION OF THE INVENTION

The compounds useful in the present methods are selected from Formula (I) hereinbelow:

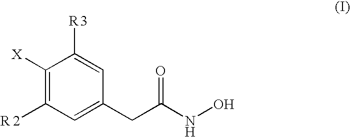

wherein:

X is selected from the group consisting of —C(O)OC₁₋₃alkyl, —OR1, —NR1R6, —C(O)NR1R6, and —C(O)R6;

R1 is selected from the group consisting of hydrogen, C₁₋₆alkyl, unsubstituted or substituted by one or more moiety selected from the group consisting of alcohol, ether, amine, amide and carboxylic acid moieties, Ar, —C₁₋₂alkylAr, C₀₋₂alkylpiperidin-4-yl, substituted on nitrogen with R7, and C₀₋₂alkylpyrrolidin-3-yl, substituted on nitrogen with R7;

Ar is selected from the group consisting of phenyl, furyl, pyridyl, thienyl, thiazolyl, isothiazolyl, pyrazolyl, triazolyl, tetrazolyl, imidazolyl, benzofuranyl, indolyl, thiazolidinyl, isoxazolyl, oxadiazolyl, thiadiazolyl, pyrrolyl, and pyrimidyl, all of which may be unsubstituted or substituted by one or more R4 or R5 groups; or R1 and R6 taken together may constitute a 5 or 6 member cyclic system which may contain an O or an optionally substituted N;

R2 is selected from the group consisting of I, Br, Cl, isopropyl and tert-butyl;

R3 is selected from the group consisting of H, I, Br, Cl, isopropyl, tert-butyl and Z-R8;

Z is selected from the group consisting of O, N, —NC(O), —C(O)N, —SO₂N, —CONHSO₂ and —CH₂;

R4 and R5 are independently selected from the group consisting of hydrogen, —OR6, —CN, F, Cl, Br, I, —CO₂H, —C(O)NR1R6, —NR6COR6, —NH₂, and —C₁₋₄alkyl, which may be unsubstituted or substituted by one or more moiety selected from the group consisting of alcohol, amine, amide and carboxylic acid;

R6 is H, or —CH₃;

R7 is selected from the group consisting of hydrogen, —C₁₋₄acyl and —C₁₋₄alkoxycarbonyl;

R8 is selected from the group consisting of —C₁₋₄alkyl, unsubstituted or substituted by one or more moiety selected from the group consisting of alcohol, amine, amide and carboxylic acid.

As used herein, “alkyl” refers to an optionally substituted hydrocarbon group joined together by carbon-carbon bonds. The allyl hydrocarbon group may be linear, branched or cyclic, saturated or unsaturated. Preferably, the group is linear. Preferably, the group is saturated. Preferred alkyl moieties are C₁₋₄ alkyl.

As used herein, “aryl” refers to an optionally substituted aromatic group with at least one ring having a conjugated pi-electron system, containing up to two conjugated or fused ring systems. “Aryl” includes carbocyclic aryl, heterocyclic aryl and biaryl groups, all of which may be optionally substituted. Preferred aryl moieties are phenyl, unsubstituted, monosubstituted, disubstituted or trisubstituted.

Preferred compounds useful in the present invention are selected from the group consisting of:

2-(3-Chloro-4-cyclopropylmethoxyphenyl)-N-hydroxyacetamide;

N-Hydroxy-2-(4-hydroxy-3,5-diiodophenyl)acetamide;

2-(4-Benzyloxy-3,5-diiodophenyl)-N-hydroxyacetamide;

2-(3,5-Diiodo-4-phenoxyphenyl)-N-hydroxyacetamide;

2-(3,5-Diiodo-4-methoxyphenyl)-N-hydroxyacetamide;

N-Hydroxy-2-(3,4,5-trimethoxyphenyl)acetamide;

2-(3,5-Di-tert-butyl-4-methoxyphenyl)-N-hydroxyacetamide;

2-(3,5-Di-tert-butyl-4-hydroxyphenyl)-N-hydroxyacetamide;

2-(3-Iodo-4-methoxy-phenyl)-N-hydroxyacetamide and

2-(4-Ethylamino-3,5-diiodophenyl)-N-hydroxyacetamide.

More preferred compounds useful in the present invention are selected from the group consisting of:

N-Hydroxy-2-[3,5-diiodo-4-(4-hydroxyphenoxy)phenyl]acetamide;

2-{4-[4-(2-Diethylaminoethoxy)phenoxy]-3,5-diiodophenyl}-N-hydroxyacetamide;

N-Hydroxy-2-[4-(4-hydroxyphenoxy)-3-iodophenyl]acetamide;

N-Hydroxy-2-(4-amino-3,5-diiodophenyl)acetamide;

N-Hydroxy-2-[3,5-diiodo-4-(4-methoxyphenoxy)phenyl]acetamide;

N-Hydroxy-2-(3,5-dichloro-4-methoxyphenyl)acetamide and

N-Hydroxy-2-(3,5-dichloro-4-phenoxyphenyl)acetamide.

Also included in the present invention are pharmaceutically acceptable salts and complexes. The compounds of the present invention may contain one or more asymmetric carbon atoms and may exist in racemic and optically active forms. All of these compounds and diastereomers are contemplated to be within the scope of the present invention.

The compounds and processes of the present invention will be better understood in connection with the following synthetic schemes, which are merely illustrative of the methods by which the compounds of the invention may be prepared and are not intended to limit the scope of the invention as defined in the appended claims.

Compounds of the formula (I) in which R4 is alkoxy or hydroxyl are prepared by the methods described in Scheme 1.

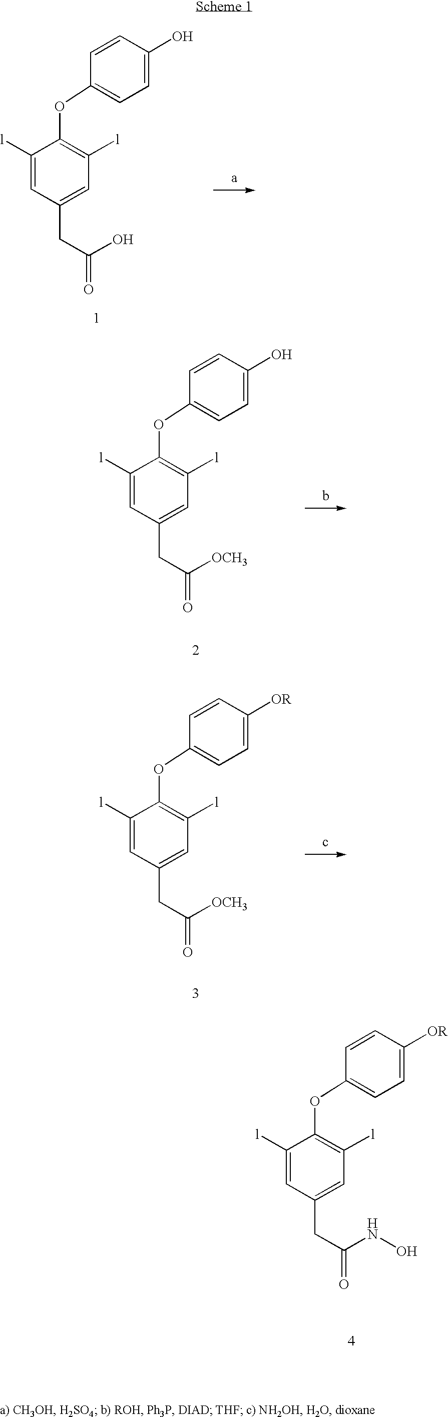

An appropriately substituted phenylacetic acid, such as 3,5-diiodothyroacetic acid 1-Scheme 1, may be esterified by refluxing in an alcohol, such as methanol, with a catalytic amount of an acid, such as sulfuric acid, to provide an ester, such as 2-Scheme 1. A phenol, such as 2-Scheme 1, may be alkylated under Mitsunobu conditions using reagents, such as triphenylphosphine, diisopropyl azodicarboxylate, and an alcohol, such as diethylaminoethanol, to provide an ether, such as 3-Scheme 1. A hydroxamic acid, such as 4-Scheme 1, may be prepared from an ester, such as 3-Scheme 1 by treatment with aqueous hydroxylamine in a solvent such as dioxane.

Compounds of the formula (I) in which R2 is iodine and R3 is hydrogen are prepared by the methods described in Scheme 2.

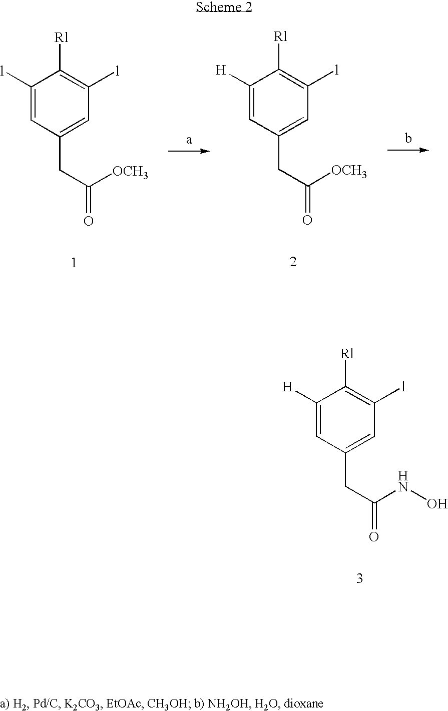

A monoiodophenylacetic ester, such as 2 Scheme 2, may be prepared by hydrogenolysis of a diiodophenylacetic ester, such as methyl 3,5-diiodo-4-methoxyphenylacetic acid 1-Scheme 2. A hydroxamic acid, such as 3-Scheme 2 may be prepared from an ester, such as 2-Scheme 2, by treatment with aqueous hydroxylamine in a solvent, such as dioxane.

Compounds of the formula (I) in which R1 is methoxy are prepared by the methods described in Scheme 3.

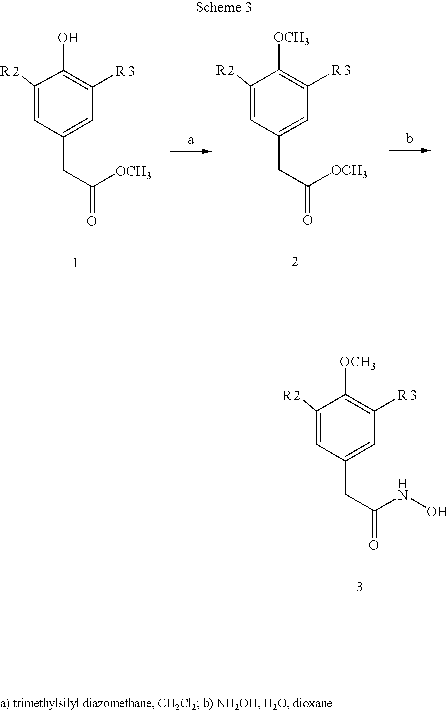

An appropriately substituted phenol, such as 1-Scheme 3, may be methylated by treatment with trimethylsilyl diazomethane in a solvent, such as dichloromethane. A hydroxamic acid, such as 3-Scheme 3, may be prepared from an ester, such as 2-Scheme 3, by treatment with aqueous hydroxylamine in a solvent such as dioxane.

Compounds of the formula (I) in which R1 is aryloxy are prepared by the methods described in Scheme 4.

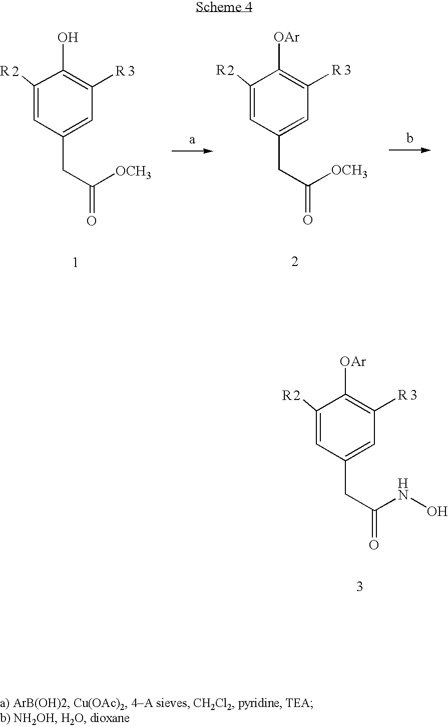

Treatment of a phenol, such as 1-Scheme 4 with an aryl boronate, such as benzene boronic acid with copper acetate, pyridine, triethyl amine and 4A sieves will provide a biaryl ether, such as 2-Scheme 4. A hydroxamic acid, such as 3-Scheme 4 may be prepared from an ester, such as 2-Scheme 4 by treatment with aqueous hydroxylamine in a solvent such as dioxane.

Compounds of the formula (I) in which R1 is NH2 are prepared by the methods described in Scheme 5.

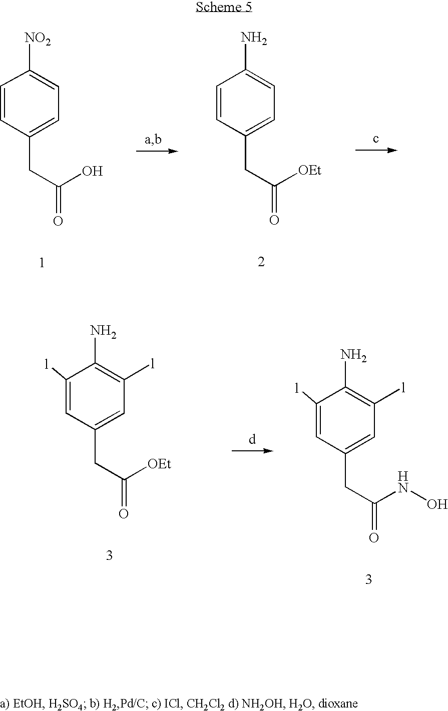

An appropriately substituted nitrophenylacetic acid, such as 1-Scheme 5, may be refluxed in an alcohol, such as ethanol, with a catalytic amount of an acid, such as sulfuric acid, to provide an ester. This ester may be reduced under a hydrogen atmosphere with a catalyst, such as palladium on carbon, to yield an amino ester, such as 2-Scheme 5. Treatment of the amino ester with iodine monochloride in a solvent, such as dichloromethane, can provide a diiodoamino ester, such as 3-Scheme 5. A hydroxamic acid, such as 3-Scheme 5 may be prepared from an ester, such as 2-Scheme 5 by treatment with aqueous hydroxylamine in a solvent such as dioxane.

The foregoing may be better understood by reference to the following examples which illustrate the methods by which the compounds of the invention may be prepared and are not intended to limit the scope of the invention as defined in the appended claims.

### EXAMPLE 1

### Preparation of N-hydroxy-2-[3,5-diiodo-4-(4-hydroxyphenoxy)phenyl]acetamide

a) Methyl 4-(4-hydroxyphenoxy)-3,5-diiodophenylacetate

A solution of 3,5-diiodothyroacetic acid (Sigma) (200 mg, 0.40 mmol) in methanol (10 ml) with sulfuric acid (10 ul) was refluxed for 3 h. HPLC confirmed complete reaction. Most of the methanol was removed in vacuo and the resultant solution diluted with ethyl acetate. This was washed with water then brine, dried (sodium sulfate) and concentrated in vacuo to afford the title compound (190 mg, 93%). ¹H NMR (400 MHz, CDCl₃): δ 7.77 (s, 2 H), 6.76 (dj=9.0 Hz, 2H), 6.67 (d j=9.0 Hz, 2H), 3.74 (s, 3H), 3.56 (s, 2H) M⁺¹=511

b) N-Hydroxy-2-[3,5-diiodo-4-(4-hydroxyphenoxy)phenyl]acetamide

A solution consisting of methyl 4-(4-hydroxyphenoxy)-3,5-diiodophenylacetate (100 mg, 0.20 mmol) in 1,4-dioxane (3 ml) and 50% aqueous hydroxylamine (2 ml) was stirred 18 h. at room temperature. Removal of all volatiles in vacuo followed by trituration in ether/hexane afforded the title compound (90 mg, 90%). ¹H NMR (400 MHz, CDCl₃): δ 7.61 (s, 2H), 6.54 (d, j=8.8 Hz, 2H), 6.41 (d, j=8.8 Hz, 1H), 3.16 (s, 2H) M⁺¹=512

### EXAMPLE 2

Preparation of 2-{4-[4-(2-diethylaminoethoxy)phenoxy]-3,5-diiodophenyl}-N-hydroxyacetamide

a) Methyl 4-[4-(2-diethylaminoethoxy)phenoxy]-3,5-diiodophenylacetate.

To an ice-cold solution of methyl 4-(4-hydroxyphenoxy)-3,5-diiodophenylacetate (100 mg, 0.196 mmol) in THF (2 ml) with 2-(N,N-diethylamino)ethanol (52 ul, 0.39 mmol), and triphenylphosphine (53 mg, 0.2 mmol) was added diisopropyl azodicarboxylate (39 ul, 0.2 mmol). The resulting solution was stirred 18 h under an argon atmosphere. All volatiles were removed in vacuo and the residue chromatographed on silica to afford the title compound (80 mg, 96%). ¹H NMR (400 MHz, CDCl₃): δ 7.77 (s, 2 H), 6.82 (d, j=9.2 Hz, 2H), 6.70 (d, j=9.2 Hz, 2H), 4.04 (t, j=5.9 Hz, 2H), 3.74 (s, 3H), 3.56 (s, 2H), 2.92 (t, j=5.9 Hz, 2H), 2.70 (q, j=7.16, 2H), 1.10 (t, j=7.16, 3H).

b) 2-{4-[4-(2-Diethylamino-ethoxy)-phenoxy]-3,5-diiodo-phenyl}-N-hydroxy-acetamide.

The title compound was prepared using the procedure in example 1b above for 2-(3,5-diiodo-4-phenoxy-phenyl)-N-hydroxy-acetamide. (51 mg, 64%). ¹H NMR (400 MHz, CDCl₃): δ 7.71 (s, 2H), 6.73 (d, j=9.2 Hz, 2H), 6.60 (d, j=9.2 Hz, 2H), 3.96 (t, j=5.9 Hz, 2H), 3.27 (s, 2H), 2.84 (t, j=5.9 Hz, 2H), 2.61 (q, j=7.16, 2H), 1.01 (t, j=7.16, 3H) M⁺¹=611

### EXAMPLE 3

Preparation of N-hydroxy-2-[4-(4-hydroxyphenoxy)-3-iodophenyl]acetamide

a) Methyl 4-(4-hydroxyphenoxy)-3-iodo-phenylacetate

To a solution of methyl 4-(4-hydroxyphenoxy)-3,5-diiodophenylacetate (420 mg, 0.823 mmol) in ethyl acetate (12 ml) and methanol (3 ml), was added potassium carbonate (145 mg, 1.05 mmol), followed by 10% Pd/C (78 mg). The mixture was stirred three hours under an atmosphere of hydrogen then filtered through celite and purified by preparative HPLC to provide (120 mg, 38%) as a white wax. ¹H NMR (400 MHz, CDCl₃): δ 7.74 (d, j=2 Hz, 1H), 7.14 (dd, j=6.4 Hz, j=2 Hz, 1H), 6.89 (d, j=8.8 Hz, 2H), 6.80(d, j=8. Hz, 2H), 6.69 (d, j=8.4 Hz, 2H), 3.70 (s, 3H), 3.55 (s, 2H). M⁺¹=385.

b) N-Hydroxy-2-[4-(4-hydroxyphenoxy)-3-iodophenyl]acetamide

A solution consisting methyl 4-(4-hydroxyphenoxy)-3-iodophenylacetate (78 mg, 0.203 mmol) in 1,4-dioxane (2 ml) and 50% aqueous hydroxylamine (2.0 ml) was stirred for 18 h at room temperature. Removal of all volatiles in vacuo followed by purification by preparative HPLC afforded the title compound (49 mg, 63%) as a white solid. ¹H NMR (400 MHz, CD₃OD): δ 7.79 (d, j=2 Hz, 1H), 7.19 (dd, j=6.4 Hz, j=2 Hz, 1H), 6.79 (q, j=6.0 Hz, 4H), 6.67 (d, j=8.4 Hz, 2H), 3.34 (s, 2H). M⁺¹=386.

### EXAMPLE 4

Preparation of N-hydroxy-2-(4-amino-3,5-diiodophenyl)acetamide:

a) Ethyl 4-aminophenylacetate

A solution of 4-nitrophenylacetic acid (5.0 g, 28 mmol) in a mixture of ethanol (100 ml) and conc. H₂SO₄ (1 ml) was refluxed for 12 h. The solution was cooled, 5% Pd/C (1.0 g) was added, and the mixutre was hydrogenated at 1 atmosphere for 2 h, at which time tlc analysis indicated that the reaction was complete. The reaction mixture was purged of H₂, the catalyst was filtered, and the filtrate was concentrated. The residue was dissolved in Et₂O, washed with aqueous NaHCO₃, dried, and the solvent removed, to provide the title compound (4.7 g, 95%). ¹H NMR (400 MHz, CDCl3): δ 1.26 (t, 3H), 3.51 (s, 2H), 3.62 (s, broad, 2H), 4.16 (q, 2H), 6.65 (d, 2H), 7.08 (d, 2H)

b) Ethyl 4-amino-3,5-diiodophenylacetate

A solution of ethyl 4-aminophenylacetate (1.0 g, 5.6 mmol) in CH₂Cl₂ (75 ml) was treated with a solution of iodine monochloride (1M in CH₂Cl₂, 16.7 mL), and the reaction was stirred for 4 h. Aqueous NaHSO₃ was added, the layers seperated, and the organic layer washed with aqueous NaHCO₃, H₂O, dried and the solvent evaporated. The residue was recrystallized from EtOH and gave the titled product (720 mg, 30%). ¹H NMR (400 MHz, CDCl3): δ 1.19 (t, 3H), 3.34 (s, 2H), 4.07 (q, 2H), 4.50 (s,broad, 2H), 7.49 (s, 2H)

c) N-hydroxy-2-(4-amino-3,5-diiodophenyl)acetamide

A solution of ethyl 4-amino-3,5-diiodophenylacetate (59 mg, 0.14 mmol) in dioxane (2 mL) was treated with NH₂OH (50% aqueous solution, 1 ml) and stirred for 3 d. The solvents were completely evaporated, and the residue was recrystallized from a mixture of MeOH and Et₂O and gave the title compound (21 mg, 36%). ¹H NMR (400 MHz, DMSO-D₆): δ 3.08 (s, 2H), 4.92 (s,broad, 2H), 7.53 (s, 2H), 8.79 (s, broad, 1H), 10.56 (s, broad, 1H)

### EXAMPLE 5

Preparation of N-hydroxy-2-[3,5-diiodo-4-(4-hydroxyphenoxy)phenyl]acetate.

a) Methyl 4-(4-methoxyphenoxy)-3,5-diiodophenylacetate

To a solution of methyl 4-(4-hydroxyphenoxy)-3,5-diiodophenylacetate (100 mg, 0.20 mmol) in dichloromethane (1.2 ml) and methanol (0.3 ml) was added dropwise trimethylsilyl diazomethane (0.5 ml of 2M solution in hexane, 0.98 mmol). The reaction mixture was stirred for 18 h and then evaporated to dryness to afford the title compound, 100 mg, as a yellow oil. M⁺¹=525.

b) N-Hydroxy-2-[3,5-diiodo4-(4-hydroxyphenoxy)phenyl]acetate

A solution consisting the above crude methyl 4-(4-methoxyphenoxy)-3,5-diiodophenylacetate in 1,4-dioxane (2.5 ml) and 50% aqueous hydroxylamine (1.5 ml) was stirred for 18 h at room temperature. Removal of all volatiles in vacuo followed by purification by preparative HPLC afforded the title compound (40 mg, 39%) as a white solid. ¹H NMR (400 MHz, CDCl₃): δ 7.77 (s, 2H), 6.83 (dd, j=9.2 Hz, j=2 Hz, 2H), 6.72 (dd, j=9.2 Hz, j=2 Hz, 2H), 3.78 (s, 3H), 3.74 (s, 3H), 3.57 (s, 2H). M⁺¹=526.

### EXAMPLE 6

Preparation of N-hydroxy-2-(3,5-dichloro-4-methoxyphenyl)acetamide.

a) Methyl 3,5-dichloro-4-methoxyphenylacetate

To a solution of methyl 3,5-dichloro-4-hydroxyphenylacetate (100 mg, 0.42 mmol) in dichloromethane (2 ml) and methanol (0.5 ml) was added dropwise trimethylsilyl diazomethane (0.84 mmol, 0.42 ml of 2M solution in hexane). The reaction mixture was stirred for 1.5 hours and then evaporated to dryness to afford the title compound, 105 mg, (100%) as a yellow oil. ¹H NMR (400 MHz, CDCl₃): δ 3.53 (s, 2H), 3.72 (s, 3H), 3.88 (s, 3H), 7.21 (s, 2H).

b) N-Hydroxy-2-(3,5-dichloro-4-methoxy-phenyl)acetamide.

A solution consisting methyl 3,5-dichloro-4-methoxy-phenylacetate (105 mg, 0.42 mmol) in 1,4-dioxane (2 ml) and 50% aqueous hydroxylamine (1.5 ml) was stirred 18 h at room temperature. Removal of all volatiles in vacuo followed by purification by preparative HPLC afforded the title compound (48 mg, 46%) as a off-white solid. ¹H NMR (400 MHz, DMSO): δ 3.29 (s, 2H), 3.81 (s, 3H), 7.37 (s, 2H), 8.91 (s, 1H), 10.66 (s, 1H). M⁺¹=250.

### EXAMPLE 7

Preparation of N-hydroxy-2-(3,5-dichloro-4-phenoxyphenyl)acetamide

a) Methyl 3,5-dichloro-4-phenoxyphenylacetate

To a flask containing of dichloromethane (9.23 ml) was added powdered 4 A sieves (2.2 g, activated at 500° C. for 8 h), phenylboronic acid (563 mg, 4.62 mmol), methyl 3,5-dichloro-4-hydroxyphenylacetate (217 mg, 0.923 mmol), copper (II) acetate (168 mg, 0.923 mmol), pyridine (0.37 ml, 4.62 mmol) and triethylamine (0.64 ml, 4.62 mmol). The reaction flask was fitted with a drying tube and stirred overnight. Filtration through celite and removal of volatiles in vacuo followed by column chromatography (silica, 10% ethyl acetate in hexane) and preparative HPLC, provided methyl 3,5-dichloro-4-phenoxyphenylacetate (105 mg, 37%) as colorless oil. ¹H NMR (400 MHz, CDCl₃): δ 7.33 (s, 2H), 7.29 (t, j=7.8 Hz, 2H), 7.05 (t, j=7.2 Hz, 1H), 6.83 (d, j=8.4 Hz, 2H), 3.75 (s, 3H), 3.60 (s, 2H). M⁺¹=311.

b) N-hydroxy-2-(3,5-dichloro-4-phenoxyphenyl)acetamide

A solution consisting of methyl 3,5-dichloro-4-phenoxyphenylacetate (105 mg, 0.33 mmol) in 1,4-dioxane (3 ml) and 50% aqueous hydroxylamine (2 mL) was stirred 18 h at room temperature. Removal of all volatiles in vacuo followed by purification by preparative HPLC afforded the title compound (54 mg, 52%) as white solid. ¹H NMR (400 MHz, DMSO): δ 10.70 (s, 1H), 8.95 (s, 1H), 7.52 (s, 2H), 7.35 (t, j=7.8 Hz, 2H), 7.08 (t, j=7.2 Hz, 1H), 6.80 (d, j=8.3 Hz, 2H), 3.38 (s, 2H). M⁺¹=312

With appropriate manipulation and protection of any chemical functionality, synthesis of the remaining compounds of Formula (I) is accomplished by methods analogous to those above and to those described in the Experimental section.

In order to use a compound of the Formula (I) or a pharmaceutically acceptable salt thereof for the treatment of humans and other mammals it is normally formulated in accordance with standard pharmaceutical practice as a pharmaceutical composition.

The present compounds are useful for the treatment of bacterial infections including but not limited to respiratory tract infections and/or Gram positive infections.

Compounds of Formula (I) and their pharmaceutically acceptable salts may be administered in a standard manner for antibiotics, for example orally, parenterally, sub-lingually, dermally, transdermally, rectally, via inhalation or via buccal administration.

Compositions of Formula (I) and their pharmaceutically acceptable salts which are active when given orally can be formulated as syrups, tablets, capsules, creams and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

Each dosage unit for oral administration contains suitably from 0.1 mg to 500 mg/Kg, and preferably from 1 mg to 100 mg/Kg, and each dosage unit for parenteral administration contains suitably from 0.1 mg to 100 mg/Kg, of a compound of Formula (I) or a pharmaceutically acceptable salt thereof calculated as the free acid. Each dosage unit for intranasal administration contains suitably 1-400 mg and preferably 10 to 200 mg per person. A topical formulation contains suitably 0.01 to 5.0% of a compound of Formula (I).

The daily dosage regimen for oral administration is suitably about 0.01 mg/Kg to 40 mg/Kg, of a compound of Formula(I) or a pharmaceutically acceptable salt thereof calculated as the free acid. The daily dosage regimen for parenteral administration is suitably about 0.001 mg/Kg to 40 mg/Kg, of a compound of Formula (I) or a pharmaceutically acceptable salt thereof calculated as the free acid, the daily dosage regimen for intranasal administration and oral inhalation is suitably about 10 to about 500 mg/person. The active ingredient may be administered from 1 to 6 times a day, sufficient to exhibit the desired activity.

No unacceptable toxicological effects are expected when compounds of the present invention are administered in accordance with the present invention.

The biological activity of the compounds of Formula (I) are demonstrated by the following test:

Biological Assay:

S. Aureus or E. Coli PDF activity is measured at 25° C., using a continuous enzyme-linked assay developed by Lazennec & Meinnel, (1997) “Formate dehydrogenase-coupled spectrophotometric assay of peptide deformylase” Anal. Biochem. 244, pp. 180-182, with minor modifications. The reaction mixture is contained in 50 uL with 50 mM potassium phosphate buffer (pH7.6), 15 mM NAD, 0.25 U formate dehydrogenase. The substrate peptide, f-Met-Ala-Ser, is included at the K_{M} concentration. The reaction is triggered with the addition of 10 nM Def1 enzyme, and absorbance is monitored for 20 min at 340 nm.

Antimicrobial Activity Assay

Whole-cell antimicrobial activity was determined by broth microdilution using the National Committee for Clinical Laboratory Standards (NCCLS) recommended procedure, Document M7-A4, “Methods for Dilution Susceptibility Tests for Bacteria that Grow Aerobically” (incorporated by reference herein). The compound was tested in serial two-fold dilutions ranging from 0.06 to 64 mcg/ml. A panel of 12 strains were evaluated in the assay. This panel consisted of the following laboratory strains: Staphylococcus aureus Oxford, Streptococcus pneumoniae R6, Streptococcus pyogenes CN10, Enterococcus faecalis I, Haemophilus influenzae Q1, Escherichia coli DC0, E. coli EES, E. coli 7623 (AcrAB+) E. coli 120 (AcrAB−) Klebsiella pneumoniae E70, Pseudomonas aeruginosa K799wt and Candida albicans GRI 681. The minimum inhibitory concentration (MIC) was determined as the lowest concentration of compound that inhibited visible growth. A mirror reader was used to assist in determining the MIC endpoint.

All publications, including but not limited to patents and patent applications cited in this specification are herein incorporated by reference as if each individual publication were specifically and individually indicated to be incorporated by reference as though fully set forth.

What is claimed is:

1. A compound according to formula (I): 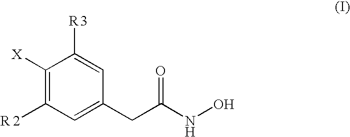
wherein:
X is selected from the group consisting of —C(O)OC₁₋₃alkyl, —OR1, —NR1R6, —C(O)NR1R6, and —C(O)R6;
R1 is selected from the group consisting of hydrogen, C₁₋₆alkyl, unsubstituted or substituted by one or more moiety selected from the group consisting of alcohol, ether, amine, amide and carboxylic acid moieties, Ar, —C₁₋₂alkylAr, C₀₋₂alkylpiperidin-4-yl, substituted on nitrogen with R7, and C₀₋₂alkylpyrrolidin-3-yl, substituted on nitrogen with R7;
Ar is selected from the group consisting of phenyl, furyl, pyridyl, thienyl, thiazolyl, isothiazolyl, pyrazolyl, triazolyl, tetrazolyl, imidazolyl, benzofuranyl, indolyl, thiazolidinyl, isoxazolyl, oxadiazolyl, thiadiazolyl, pyrrolyl, and pyrimidyl, all of which may be unsubstituted or substituted by one or more R4 or R5 groups; or R1 and R6 taken together may constitute a 5 or 6 member cyclic system which may contain an O or an optionally substituted N;
R2 is selected from the group consisting of I, Br, Cl, isopropyl and tert-butyl;
R3 is selected from the group consisting of H, I, Br, Cl, isopropyl, tert-butyl and Z-R8;
Z is selected from the group consisting of O, N, —NC(O), —C(O)N, —SO₂N, —CONHSO₂ and —CH₂;
R4 and R5 are independently selected from the group consisting of hydrogen, —OR6, —CN, F, Cl, Br, I, —CO₂H, —C(O)NR1R6, —NR6COR6, —NH₂, and —C₁₋₄alkyl, which may be unsubstituted or substituted by one or more moiety selected from the group consisting of alcohol, amine, amide and carboxylic acid;
R6 is H, or —CH₃;
R7 is selected from the group consisting of hydrogen, —C₁₋₄acyl and —C₁₋₄alkoxycarbonyl;
R8 is selected from the group consisting of —C₁₋₄alkyl, unsubstituted or substituted by one or more moiety selected from the group consisting of alcohol, amine, amide and carboxylic acid.

2. A compound according to claim 1 selected from the group consisting of:
2-(3-Chloro-4-cyclopropylmethoxyphenyl)-N-hydroxyacetamide;
N-Hydroxy-2-(4-hydroxy-3,5-diiodophenyl)acetamide;
2-(4-Benzyloxy-3,5-diiodophenyl)-N-hydroxyacetamide;
2-(3,5-Diiodo-4-phenoxyphenyl)-N-hydroxyacetamide;
2-(3,5-Diiodo-4-methoxyphenyl)-N-hydroxyacetamide;
N-Hydroxy-2-(3,4,5-trimethoxyphenyl)acetamide;
2-(3,5-Di-tert-butyl-4-methoxyphenyl)-N-hydroxyacetamide;
2-(3,5-Di-tert-butyl-4-hydroxyphenyl)-N-hydroxyacetamide;
2-(3-Iodo-4-methoxy-phenyl)-N-hydroxyacetamide and
2-(4-Ethylamino-3,5-diiodophenyl)-N-hydroxyacetamide.

3. A compound according to claim 2 selected from the group consisting of:
N-Hydroxy-2-[3,5-diiodo-4-(4-hydroxyphenoxy)phenyl]acetamide;
2-{4-[4-(2-Diethylaminoethoxy)phenoxy]-3,5-diiodophenyl}-N-hydroxyacetamide;
N-Hydroxy-2-[4-(4-hydroxyphenoxy)-3-iodophenyl]acetamide;
N-Hydroxy-2-(4-amino-3,5-diiodophenyl)acetamide;
N-Hydroxy-2-[3,5-diiodo-4-(4-methoxyphenoxy)phenyl]acetamide;
N-Hydroxy-2-(3,5-dichloro-4-methoxyphenyl)acetamide and
N-Hydroxy-2-(3,5-dichloro-4-phenoxyphenyl)acetamide.

4. A method of treating a bacterial infection by administering to a subject in need of treatment, compound according to claim 1.

5. A method according to claim 4 selected from the group consisting of:
2-(3-Chloro-4-cyclopropylmethoxyphenyl)-N-hydroxyacetamide;
N-Hydroxy-2-(4-hydroxy-3,5-diiodophenyl)acetamide;
2-(4-Benzyloxy-3,5-diiodophenyl)-N-hydroxyacetamide;
2-(3,5-Diiodo-4-phenoxyphenyl)-N-hydroxyacetamide;
2-(3,5-Diiodo-4-methoxyphenyl)-N-hydroxyacetamide;
N-Hydroxy-2-(3,4,5-trimethoxyphenyl)acetamide;
2-(3,5-Di-tert-butyl-4-methoxyphenyl)-N-hydroxyacetamide;
2-(3,5-Di-tert-butyl-4-hydroxyphenyl)-N-hydroxyacetamide;
2-(3-Iodo-4-methoxy-phenyl)-N-hydroxyacetamide and
2-(4-Ethylamino-3,5-diiodophenyl)-N-hydroxyacetamide.

6. A method of treating a bacterial infection according to claim 5 selected from the group consisting of respiratory tract infection, and Gram+ TPP.
